SEQUENCE LISTING (7 sequences)

SEQ ID NO: 1 - protein, 9 aa, Homo sapiens
KVAELVHFL

SEQ ID NO: 2 - protein, 9 aa, Homo sapiens
FLWGPRALV

SEQ ID NO: 3 - protein, 9 aa, Homo sapiens
SLLMWITQC

SEQ ID NO: 4 - protein, 9 aa, Homo sapiens
VLDFAPPGA

SEQ ID NO: 5 - protein, 9 aa, Homo sapiens
RMFPNAPYL

SEQ ID NO: 6 - protein, 9 aa, Homo sapiens
SLGEQQYSV

SEQ ID NO: 7 - protein, 9 aa, Homo sapiens
CMTWNQMNL

(19) United States
(12) Patent Application Publication
(10) Pub. No.: US 2015/0166955 A1
(43) Pub. Date: Jun. 18, 2015

(54) METHOD FOR LOADING OF DENDRITIC CELLS WITH CLASS I ANTIGENS

(71) Applicant: DCPRIME B.V., Leiden (NL)

(72) Inventors: Sandra Van Wetering, Leidschendam (NL); Adriana Marie Kruisbeek, Amsterdam (NL)

(21) Appl. No.: 14/412,328

(22) PCT Filed: Jul. 2, 2013
(86) PCT No.: PCT/EP2013/063961

§ 371 Date: Dec. 31, 2014

(30) Foreign Application Priority Data
Jul. 2, 2012 (NL) 2009102

(51) Int. Cl. C12N 5/0784; A01N 1/02; A61K 39/00; C12N 13/00

(52) U.S. Cl. CPC C12N 5/0639; C12N 13/00; A01N 1/021; A61K 39/0011; C12N 2506/11; C12N 2501/22; C12N 2501/2304; C12N 2501/25; C12N 2501/2301; C12N 2501/2306; A61K 2039/5154

(57) ABSTRACT

Disclosed is a method to obtain dendritic cells loaded with an exogenously added antigen. The method is in particular applicable to human cell lines that may be used in the production of dendritic cell vaccines, e.g. to be used in the treatment of cancer. In the method the antigen to be loaded on the cells is added to the cells at particular stages of the development of the dendritic cells. A pharmaceutical composition comprising the obtained cells is also disclosed.

### FIELD OF THE INVENTION

The invention is in the field of medical treatments. It provides means and methods for providing antigen presenting cells such as dendritic cells, for use in a medical therapy. In particular it provides methods for the preparation of dendritic cells loaded with an antigenic peptide that can be used as an off-the-shelf vaccine against a variety of illnesses such as cancer, autoimmune diseases and other diseases as further detailed herein. Dendritic cells produced in the method according to the invention are capable of stimulating specific cytotoxic T cells to attack cells that express the antigenic peptide.

### BACKGROUND OF THE INVENTION

Dendritic cells (DCs) are the most powerful antigen presenting cells (APC) and play a pivotal role in initiating the immune response. DCs develop from hematopoietic precursor cells in the bone marrow, going through sequentially different stages of differentiation (intermediary precursor cells in blood and immature DCs in peripheral tissues and organs; Banchereau et al. 2000 Ann. Rev. Immunol. 18: 767-811).

Jacobs et al. (Horm Metab Res. 2008 Feb;40(2):99-107) provides an overview of dendritic cell subtypes and in vitro generation of dendritic cells. The article describes the identification of different DC subpopulations including phenotypical and functional differences and describes recent developments on protocols for generation of DCs. It also discloses that various cytokines and transcription factors are known to be responsible for the development of DC subpopulations. Depending on the subpopulation and the maturation state of these cells, they are either are able to induce a broad cytotoxic immune response, and therefore represent a promising tool for anticancer vaccination therapies in humans or induce immune tolerance and are important within the context of autoimmunity.

DCs can be obtained ex vivo by differentiating progenitor cells, for example CD34 positive cells, under influence of various immunostimulatory molecules. For example, murine bone marrow (BM)-derived progenitor cells could differentiate into myeloid DCs in the presence of granulocyte-macrophage colony-stimulating factor (GM-CSF). In humans, the addition of tumor necrosis factor-α (TNF-α), GM-CSF and IL-4 was shown to induce the development of DCs from purified CD34+ cells (CD34 positive cells) derived from bone marrow, cord blood (CB) and peripheral blood (PB).

In addition to GM-CSF and TNF-α, a broad spectrum of cytokines has been shown to influence DC progenitor growth and differentiation. Early acting growth factors, such as stem cell factor (SCF) and Flt-3 ligand (Flt-3L) sustain and expand the number of DC progenitors whereas IL-3 in combination with GM-CSF has been shown to enhance DC differentiation. Moreover, transforming growth factor (TGF)-beta1 potentiates in vitro development of Langerhans-type DC.

DCs are specialized in picking up and processing antigens into peptide fragments that bind to major histocompatibility complex (MHC) molecules. Located in most tissues, DC migrate from the periphery to secondary lymphoid organs such as the spleen and the lymph nodes, where antigen specific T lymphocytes recognize, through the T cell receptor, the peptide-MHC complexes presented by the DC. While other professional and non-professional APC can only stimulate activated or memory T cells, DC have the unique capacity to prime naive and quiescent T lymphocytes.

Given their pivotal role in controlling immunity, the therapeutic role of DC has been proposed for many diseases that involve T-cell activation, such as autoimmune diseases, inflammatory diseases and neoplastic disorders. For example, ex vivo pulsing (loading) with tumor antigens and the subsequent reinfusion of DC can lead to protection against tumors in animals. To address the efficacy of DC-based tumor immunotherapy strategies in humans, several clinical trials involving DC are currently in progress. Other examples of conditions that could benefit from the use of pulsed DC's are auto-immune, inflammatory and infectious diseases.

Dendritic cells may be derived from hematopoietic bone marrow progenitor cells. These progenitor cells initially transform into immature dendritic cells. These cells are characterized by high endocytic activity and low T-cell activation potential. Immature dendritic cells constantly sample the surrounding environment for pathogens such as viruses and bacteria. This is done through pattern recognition receptors (PRRs) such as the toll-like receptors (TLRs). TLRs recognize specific chemical signatures found on subsets of pathogens. Immature dendritic cells may also phagocytize small quantities of membrane from live own cells, in a process called nibbling. Once they have come into contact with a presentable antigen, they become activated into mature dendritic cells and begin to migrate to the lymph node.

Immature dendritic cells are known to phagocytose pathogens and degrade their proteins into small pieces and upon maturation present those fragments at their cell surface using MHC molecules in a process called “antigen processing”. Simultaneously, they upregulate cell-surface receptors that act as co-receptors in T-cell activation such as CD80 (B7.1), CD86 (B7.2), and CD40 greatly enhancing their ability to activate T-cells. They also upregulate CCR7, a chemotactic receptor that induces the dendritic cell to travel through the blood stream to the spleen or through the lymphatic system to a lymph node. Here they act as antigen-presenting cells: they activate helper T-cells and killer T-cells as well as B-cells by presenting them with antigens derived from the pathogen, alongside non-antigen specific costimulatory signals.

As mentioned herein, mDC may arise from monocytes, white blood cells which circulate in the body and, depending on the right signal, can turn into either dendritic cells or macrophages. The monocytes in turn are formed from stem cells in the bone marrow. Monocyte-derived dendritic cells can be generated in vitro from peripheral blood mononuclear cells (PBMCs). Plating of PBMCs in a tissue culture flask permits adherence of monocytes. Treatment of these monocytes with interleukin 4 (IL- 4) and granulocyte-macrophage colony stimulating factor (GM-CSF) leads to differentiation to immature dendritic cells (iDCs) in about a week. Subsequent treatment with tumor necrosis factor (TNF) further differentiates the iDCs into mature dendritic cells (mDCs).

It is important to distinguish between the terms “loading” and “processing” in the context of expressing antigenic peptides at the surface of a DC. Peptide loading or antigen loading as used herein refers to a process wherein cells are contacted with the peptide or the antigen and wherein the peptide or the antigen is attached to the MHC complex at the surface of the cell without internalizing the antigen or the peptide. The term processing as used herein refers to the unique property of immature DCs to internalize a peptide or an antigen, process it into fragments that are then transported to the cell membrane and expressed in the context of an MHC molecule.

Dieckmann (Dieckmann et al. 2005 International Immunology 17 (5): 621-635) compared loading of antigen peptides by immature DC (im-DC) and mature DC (m-DC). Loading was for one hour. They concluded that only mature DC (m-DC) but not immature DC (im-DC) could be sufficiently loaded with exogenously added peptides and were by far superior in expanding T cell responses. These results indicate that stimulation with m-DCs is superior in terms of quantity and quality compared with im-DCs, supporting their preferred use in clinical DC trials (see also e.g. Zehn at al. (2006) Int. Immunol. 18(12): 1647-1654)./nlp

Although knowledge is accumulating with respect to how different progenitors differentiate under influence of different compounds, like cytokines to various types of DCs, and what protocols may best be used to provide loaded DCs, typically however, culturing time is still long, and yields are low.

From the above it will be clear to the person skilled in the art that there is need for further improvement of the available methods for the ex vivo production of DC-comprising vaccines from progenitor cells and loaded with exogenously added antigens. In particular there appears to be a need for methods that allow large scale production of such loaded DCs in short time periods. This would allow for sufficient material to be obtained for use in treatment of various disease conditions including cancers, auto-immune, inflammatory and infectious diseases.

### SUMMARY OF THE INVENTION

The present invention provides a method for obtaining mature dendritic cells loaded with an HLA Class I antigenic peptide, the method comprising the steps of:
a) obtaining immature dendritic cells
b) cultivating the immature dendritic cells in a culture medium in the presence of at least one compound that is capable of inducing maturation of the immature dendritic cells, and wherein during at least part of the period of the cultivating at least one exogenously added HLA Class I antigenic peptide is present in the culture medium,
c) wherein the peptide consists of 8, 9, 10 or 11 amino acids and
d) collecting the mature dendritic cells loaded with an HLA Class I antigenic peptide.

The present invention also provides a pharmaceutical composition comprising mature dendritic cells obtainable by the method described above.

The present invention also relates to such a pharmaceutical composition for use in the treatment of cancer, inflammatory and infectious diseases and autoimmune diseases. In other words, the present invention provides a method of treatment comprising the administration of a pharmaceutical composition according to the invention to a subject in need of such a treatment. The diseases that may be treated with a method according to the invention are immune diseases, such as diseases selected from the group consisting of cancer, inflammatory and infectious diseases and autoimmune diseases.

The invention also provides an in vitro method for activating cytotoxic T-cells specific for a tumor antigen, comprising the contacting of a mature DC obtained by the method according to any one of claims 1-12 with a population of T-cells, preferably CD8+ T-cells.

### DETAILED DESCRIPTION OF THE INVENTION

Throughout this application, various references are cited to describe more fully the state of the art to which this invention pertains. The disclosures of these references are hereby incorporated by reference into the present disclosure in their entirety.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), Sambrook and Russel, Molecular Cloning: A Laboratory Manual 3rd ed., Cold Spring Harbor Laboratory Press (Cold Spring Harbor, N.Y. 2001), and Janeway's Immunobiology, (8th edition New York: Garland Science; 2011) provide one skilled in the art with a general guide to many of the terms used in the present application. One skilled in the art will recognize many methods and materials similar or equivalent to those described herein, which may be used in the practice of the present invention. Indeed, the present invention is in no way limited to the methods and materials described. For purposes of the present invention, the following terms are defined below.

The person skilled in the art knows what is to be construed with the term “CD34 positive cells”. It refers to cells (primary or cell lines) naturally expressing CD34 on the cell surface and which are known to be capable to differentiate into DCs (see e.g. Reid et al. Blood 76:1139, 1990; Bernhard et al. Cancer Res 1995;55:1099-1104). It however does not refer to cells that normally do not express CD34, but have been modified, for example by the introduction of a plasmid carrying DNA encoding CD34, to express CD34.

The person skilled in the art knows that the term “compound that is capable of inducing differentiation of the cells” relates to such compound that, alone or in specific combination, can induce, when present in sufficient amounts in for example culture medium, the differentiation of dendritic precursor cells, like the above described CD34 positive cells, into or towards dendritic cells. Preferred compounds are chemical or biological compounds. Well known, non-limiting, examples include, such chemical and biological molecules which influence the differentiation of cells, such as cytokines (IL-4 (Interleukin 4), IL-6, PGE-2, TNFalpha (Tumour Necrosis Factor Alpha), TGF-beta (transforming growth factor beta), growth factors such as Granulocyte-macrophage colony-stimulating factor (GM-CSF), and surrogate molecules for cytokines or growth factors inducing a biological effect comparable to that of the stimulatory molecules themselves, e.g. antibodies, other biological molecules such as for example LPS or polyIC. (Bürdek et al. Journal of Translational Medicine 2010, 8:90; Thurner at al. Journal of Immunological Methods 223 1999, 1-15).

At the same time, the person skilled in the art is aware of methods available in the art for obtaining mature dendritic cells from, for example, the immature dendritic cells described above. For example, immature dendritic cells may be matured by adding chemical or biological compounds such as TNF-alpha, IL-6, IL-1 beta and/or prostaglandin E2, although other methods known in the art to mature immature dendritic cells may also be employed.

In order to obtain fully matured dendritic cells, the cells may, for example be further treated with at least one compound selected from the group consisting of TNF-alpha, IL-6, PGE-2 or IL-1 beta or combinations thereof. Such treatment will allow for obtaining mature dendritic cells from immature dendritic cells. The cells thus obtained are fully functional as dendritic cells as can be witnessed from the fact that the obtained cells express high levels of MHC Class I and II and CD83 which is a typical marker for mature DCs. Only mature DC have the capacity to prime an immune response (see Steinman (1991) Annu. Rev. Immunol. 9: 271-296 and Caetano (2006) Nature Reviews Immunology 6:476-483).

The skilled person understands the term “mature dendritic cell”. In contrast to immature dendritic cells, dendritic cells are characterized by the expression of the maturation marker CD83 (see e.g. Cao et al. 2005 Biochem. J. 385: 85-93). In addition, mature dendritic cells show higher expression of MHC and co-stimulatory molecules (see, e.g., Nierkens et al. (2011) Cancers 3: 2195-2212) compared to immature DC. Also mature DC have a greater capacity to migrate towards lymph node homing chemokines and a higher T-cell stimulatory capacity compared to immature DC.

The skilled person understands the term “immature dendritic cell” as a cell that is characterized by the expression of CD1a (see eg. Slom et al (2008) J. Immunol. 180: 980-987) on the surface of the cell (see also US2004265998). At the same time immature DCs have low expression of the costimulatory molecules CD80 and CD86, whereas the maturation marker CD83 is absent (or low). Upon maturation, the expression of MHC class I and II and the co-stimulatory molecules CD80, CD86 and CD83 increases. As described above, the skilled person knows how to provide for immature DCs. For example, they may be obtained from CD34+ (CD34 positive) positive stem cells or monocytes, by first differentiating the cells, followed by maturation to obtain mature DCs.

The present invention provides for a method for obtaining mature dendritic cells that are loaded with an exogenously added peptide, the method comprising the steps of:
a) obtaining immature dendritic cells
b) cultivating the immature dendritic cells in a culture medium in the presence of at least one compound that is capable of inducing maturation of said immature dendritic cells, and wherein during at least part of the period of said cultivating at least one exogenously added peptide is provided to the culture medium
c) collecting the mature dendritic cells.

As is witnessed from the examples, it was surprisingly found that, in contrast to suggestions in the art, loading of the cells with a peptide, preferably a short peptide (see below), during the maturation of the cells (i.e. in the presence of compounds inducing the maturation of the immature dendritic cells) from immature to mature DCs provided for mature DCs that where at least as efficiently loaded as where DCs that were first matured into fully mature DCs, and then loaded with such peptide.

The term “short peptide” in this context is defined as a peptide consisting of 8, 9, 10 or 11 amino acids, preferably a peptide consisting of 9 amino acids.

In addition, it was surprisingly found that loading of the maturing dendritic cell (i.e. loading during the maturation from immature to mature in the presence of at least one compound that is capable of inducing maturation of said immature dendritic cells) did not influence viability of the cells per se.

Even more importantly, it was found that loading during the maturation of the dendritic cells (from immature to mature dendritic cells) did not negatively influence the ability of the maturing dendritic cell to mature. This is evidenced by the fact that the phenotype of the mature dendritic cell, which was provided with exogenously added peptide to be loaded during the maturation, did not differ from the phenotype of a mature dendritic cell that was matured in the absence of such peptide.

Furthermore, it was surprisingly found that the specific method of loading disclosed herein (i.e. during maturation of the cells in the presence of at least one compound that is capable of inducing maturation of said immature dendritic cells) does not negatively influence its mature dendritic cells function, i.e. migration and capability for T-cell proliferation, for example as determined using a MLR assay (mixed leukocyte reaction), or activation of specific T-cells.

Consequently, the current disclosure abrogates the need to load dendritic cells after they have first been matured into fully mature dendritic cells, as suggested by, for example, Dieckmann (Dieckmann et al. 2005 International Immunology 17 (5): 621-635), which in turn reduces time to provide for mature dendritic cells loaded with an exogenously added peptide for use as a vaccine. Since mature DCs only have a limited lifespan the current method, by abolishing the time consuming step of loading matured DCs (after they have been matured), is an important improvement in the production of DCs for use in vaccines.

Moreover the current invention reduces the chance for errors and/or infections during the production of such vaccine. In addition, loading after maturation limits the amount of peptide en dendritic cells one can load in a single experiment due to the fact such loading occurs for a short period of time (e.g. 2 hrs) in a small volume (in the range of 500-3000 μl per 1-10 million cells; loaded with 20-50 microgram peptide per milliliter (see e.g. Clin cancer Res (2011)17:1984-1997 or Cancer Immunol. Immunother (2011): 60(2):249-60). In particular for dendritic cell lines, the method disclosed herein allows to load the cells during the process of maturation on a much larger scale, which improves the standardization of the cell vaccine, the quality of the vaccine as well as time to produce, e.g., a mature dendritic cell, e.g., for use as a dendritic cellvaccine. By loading dendritic cell lines with the method according to the invention, dendritic cell vaccines are provided that are very well characterized and standardized over time. Each patient is able to receive the same vaccine, without any variations between patients or over time.

As documented above, the skilled person is well able to provide for immature dendritic cells as defined herein, and for example as described in a preferred embodiment below.

In a method according to the invention, the immature dendritic cells may be cultivated using conventional and suitable media for maturing dendritic cells, e.g. as described by Masterson (Masterson A. J. (2002) Blood 100, 701-703). The skilled person knows under what conditions such cultivating must take place.

In order to induce maturation of the dendritic cells at least one compound capable of inducing maturation of said immature dendritic cells is present or may be added to the medium for growth of the cells, and in an amount that is effective in inducing said maturation. Examples of such compounds are documented herein and are known to the skilled person.

During at least part of the period of maturation (from immature DC to mature DC), at least one exogenously added peptide is provided to the medium. The exogenously added peptide may be added directly to the medium comprising the maturing dendritic cells, but also be added to the cells by replacing the medium for maturation with the same medium and now comprising the peptide that is to be loaded to the cell surface of the dendritic cells. It is not required that the peptide is provided to the culture medium from the start of the induction of maturation or until the end of the maturation. However, preferably the peptide is present until the maturation of the dendritic cells is considered complete.

The peptide is preferably an antigenic peptide, e.g. a cancer specific antigen or an antigen that plays a role in autoimmune conditions, like diabetes, or chronic infectious diseases such as HIV, or inflammatory conditions, like Rheumatoid arthritis, as documented herein, or any other antigen known in the art, and comprises the full epitope. The peptide, when presented on the cell surface of mature dendritic cells, will normally, for example, elicit a T-cell response directed against the loaded peptide. It is also preferred that the peptide is a HLA class I peptide, i.e. a peptide capable of being presented to the immune system in the context of an MHC class 1 complex. In that way, cytotoxic T-cells or CD8+ cells may be stimulated to attack cells such as tumor cells expressing the antigenic peptide.

In particular the method according to the current invention now allows for large-scale, off-the-shelf, production of such vaccine comprising dendritic cells loaded with an exogenously added peptide by abrogating the time-consuming, expensive and risky (infection) loading of dendritic cells after the have being cultivated to maturity. In addition, by abrogation of the loading after maturation step, cell yield is dramatically improved by the method according to the invention, in comparison to loading after maturation

In a further preferred embodiment, the peptide to be loaded at the surface of the dendritic cells, and which is added during maturation of the dendritic cells, is a MHC class I or MHC class II molecule, preferably a MHC class I peptide. In another embodiment, the peptide is a peptide with a length of between, and including 6-20 adjacent amino acids, preferably 6, 7, 8, 9, 10, 11, 12, 13, 14, 15, 16, 17, 18, 19, 20 adjacent amino acids, more preferably 8, 9, 10, or 11 adjacent amino acids. The use of a peptide consisting of 9 amino acids is most preferred.

It was found that in particular the loading during the maturation of dendritic cells with peptides having a length of 8, 9, 10 or 11 adjacent amino acids, provides for mature dendritic cells that display the antigen/epitope comprised in such peptide, equally efficient as when dendritic cells were first matured and loading takes place after such maturation. Peptides with a length of 9 amino acids are particularly preferred.

In one of its embodiments, the invention therefore relates to a method for obtaining mature dendritic cells loaded with an HLA Class I antigenic peptide, the method comprising the steps of:
a) obtaining immature dendritic cells
b) cultivating the immature dendritic cells in a culture medium in the presence of at least one compound that is capable of inducing maturation of said immature dendritic cells, and wherein during at least part of the period of said cultivating at least one exogenously added HLA Class I antigenic peptide is present in the culture medium,
c) wherein the peptide consists of 8, 9, 10 or 11 amino acids, and
d) collecting the mature dendritic cells loaded with an HLA Class I antigenic peptide.

A valid Tumor Associated Antigen (TAA) must be specifically expressed by the tumor and/or substantially overexpressed by the tumor relative to other normal cell types so that the given therapy preferentially targets the tumor rather than normal tissues. In addition to this, the TAA, must be presented in the context of MHC class I molecules (Yu et al, 2002) which can be recognized by TAA specific Cytotoxic T-Lymphocytes (CTL), the essential players in tumor clearance in vivo (Feltkamp et al, 1995). Short, peptides, such as peptides consisting of 9 amino acids, containing immunodominant epitopes, may be loaded directly into the MHC class I molecules of DC, without being processed or internalised. Melan-A/MART-126-35 parental or Melan-A/MART-126-35L analog epitopes, which can be presented by HLA-A2, have been widely used in melanoma immunotherapy to induce and boost in vivo CTL responses this way (Bioley et al, 2010).

Thus, there is provided for a new method of loading dendritic cells with a peptide, wherein the loading with the peptide takes place in the presence of a compound inducing maturation and said dendritic cells that have not yet fully matured (i.e. dendritic cells that respond to the compound that induces maturation of dendritic cells, for example such as documented herein, by acquiring/showing increased characteristics of mature DCs, such as expression of CD83 or other markers as documented herein). In a preferred embodiment, the loading in started at the same time as the maturation of the (fully) immature dendritic cells is started. Alternatively, the peptides are provided to the medium comprising at least one compounds that is capable of inducing maturation of said immature dendritic cells, after the maturation has started.

Although any type of immature DC may be used (e.g. using monocyte derived dendritic cells) according to the invention, it is in a preferred embodiment that the immature dendritic cells are obtained from CD34+ progenitor cells of a cell line, preferably a CD34+ cell line, preferably selected from the group consisting of KG1, THP-1, HL-60, K562, U-937 or MUTZ3, or cell lines derived thereof. In a further preferred embodiment, immature DCs are obtained from a cell line called DCOne. This cell line has been deposited at the Deutsche Sammlung von Mikroorganismen and Zellkulturen GmbH (DSMZ or German Collection of Microorganisms and Cell Cultures) at Inhoffenstrabe 7 B, 38124 Braunschweig, Germany under accession numbers DSMZ ACC3189 on Nov. 15, 2012.

For example, the immature dendritic cells can be obtained from autologous CD34 positive cells obtained from a subject. However, it is preferred the immature dendritic cells are from a (clonal) cell line, for example obtained by differentiating progenitors of such cell lines into immature DCs, for example as documented herein.

The skilled person understands the term cell line. Within the context of the current disclosure, a cell line is immortalized and has the ability to proliferate indefinitely, whereas a primary cell has a limited lifespan and limited proliferation capacities.

It is important to realize that the type of cell or cell line is less relevant as long as the cell line is a cell line capable of being matured into mature dendritic cells. Preferred examples included the cell lines KG1, THP-1, HL-60, K562, U-937 or MUTZ3, or cell lines derived thereof (see also US2004265998, Santegoets et al 2008 J Leukoc Biol 84(6): 1364). Particularly preferred is the DCOne cell line.

DCOne is a precursor cell line for dendritic cells also known as a progenitor cell line. The DCOne cell line has been derived from the peripheral blood of a patient with acute myeloid leukemia (AML) FAB M4. DCOne progenitor cells are CD34 positive (CD34 +) and CD1a negative (CD1a−) as well as CD83 negative (CD83−). Furthermore, they are low in expression or even lack a number of other receptors associated with DC differentiation and maturation, including DCSign, Langerin, CD40 and CD80. Most importantly, DCOne progenitor cells express WT-1 protein.

DCOne cells may be multiplied in conventional cell culture systems typically used for expansion of hematopoietic cell lines. A typical example of such a culture system comprises MEM-α medium containing FCS, supplemented with GM-CSF, L-Glutamine, and Penicillin/streptomycin. In more detail, the culture system may comprise MEM-α medium containing FCS (10-20%), supplemented with GM-CSF (range 20-40 IU/ml)), L-Glutamine (2 mM), and Penicillin/Streptomycin (100 IU/ml; 100 μg/ml). A particular suitable culture system may comprise MEM-α medium containing 20% FCS, supplemented with GM-CSF (25 IU/ml), L-Glutamine (2 mM), and Penicillin/streptomycin.

The DCOne cells may be stored at a concentration of 2.5-40 *10E6 cells/ml in 12.5% DMSO and 87.5% FCS in nitrogen. The process of multiplying DCOne cells is also referred to herein as proliferation of the cells.

To generate functional, mature dendritic cells from DCOne progenitor cells, DCOne progenitor cells have to undergo a process of stimulation with the appropriate stimulatory molecules. The skilled person will be aware of the metes and bounds of this process of stimulation also known as differentiation and maturation.

Tsuchiya and co-workers first described the THP-1 cell line as a human leukemia cell line with distinct monocytic characteristics such as lysozyme production and phagocytosis capacity. THP-1 cells have been demonstrated to acquire DC properties upon stimulation with cytokines, the DC differentiation capacity of THP-1 cells is relatively low, as generally less than 5% of THP-1 cells express the classic myeloid DC marker CD1a after differentiation. The inclusion of calcium ionophores (CI resulted in complete differentiation and instant maturation of the THP-1 cells, expressing high levels of CD80, CD86, CD40, and CD83, displaying increased, allogeneic T cell-stimulatory capacity and markedly decreased receptor-mediated endocytosis capacity within 24 h.

KG-1 is a cytokine-responsive, CD34+ myelomonocytic cell line derived from a patient with erythroleukemia undergoing myeloblastic relapse]. KG-1 cells have been described to acquire DC-like properties upon stimulation with cytokines or PMA±CI and differentiation was accompanied by distinct expression of the DC maturation marker CD83, indicating instant maturation induction.

The MUTZ-3 cell line (available from the Deutsche Sammlung von Mikro-organismen and Zellkulturen, Braunschweig, Germany) is derived from the peripheral blood of a patient with acute myelomonocytic leukemia. In the human dendritic cell line MUTZ3, cells differentiate to DC's under influence of cytokines like GM-CSF, IL-4 and TNF-alpha, whereas GM-CSF, TGF-beta1 and TNF-alpha also potentiates in vitro development of Langerhans-type DC's. Importantly, MUTZ-3-derived IDC and LC could also be matured further under the influence of cytokines or CD40 ligation, resulting in up-regulation of co-stimulatory and adhesion molecules CD80, CD86, CD40, CD54, and HLA-DR and de novo expression of CD83.

In another preferred embodiment of the method according to the invention, the CD34 positive cells are CD34 positive DCOne cells, MUTZ3 cells, CD34 positive human cells or CD34 positive tumor cells. It has been found that in particular these cells can advantageously be utilized in the method according to the invention.

The current method is suitable for cell lines, although, as discussed above, also autologous cells, e.g. peripheral blood monocytes, may be used. Cell lines that can be differentiated and matured into functional DCs provide for standardized and off-the-shelf availability of DC vaccine, for example for use in the treatment of cancer. Standardized since protocols of expansion, differentiation, loading, maturation, and producing the vaccine can be fully optimized to the particular cell line, and since the starting material for producing the vaccine is identical in time. Off-the-shelf availability of such vaccine allow for superior quality and quality control over vaccine produced from autologous obtained material, with its inherent variability between subjects, and the advantage of being able to start treatment without the need to first produce a vaccine (as is for autologous material), without the danger of not being able to provide for sufficient vaccine (as is for autologous material derived vaccine).

In a further preferred embodiment the antigen peptide is selected from the group consisting of antigen peptide of a tumor antigen derived from WT-1, NY-ESO, MAGE-A3, MELAN A/Mart-1, NY-ESO, PRAME, RHAMM, PSA, PSMA, Her2Neu and/or MUC-1. In a preferred embodiment, the peptide sequence is selected from the group consisting of SEQ ID NO: 1-7.

Non-limiting examples of suitable peptides for use in the current invention include: Mage-A3: KVAELVHFL (112-120; SEQ ID NO: 1) and FLWGPRALV (271-279; SEQ ID NO: 2), and heteroclytic variants, NY-ESO: SLLMWITQC (157-165; SEQ ID NO: 3) and heteroclytic variants, WT1: VLDFAPPGA (37; SEQ ID NO: 4), RMFPNAPYL (126-134; SEQ ID NO: 5), SLGEQQYSV (187; SEQ ID NO: 6) and CMTWNQMNL (235; SEQ ID NO: 7) and heteroclytic variants.

In a further embodiment, the invention relates to a method as described above, wherein a combination of different peptides, e.g. antigen peptides, is provided to the medium.

It was found that, in particular when using immature DCs obtained from cell lines, such as those exemplified herein, more than one (type of) peptide may be added to the medium in order to be loaded on the cell surface of the dendritic cell. Loading of more than one (type of) peptide, either directed to the same target protein (e.g. Her2Neu) or directed to different target proteins, during cultivating in the presence of a compound that is capable of inducing maturation in immature dendritic cells (and is present at a concentration that induces maturation of the immature dendritic cells) allows for the provision of mature dendritic cells loaded with different types of peptides.

Alternatively, if so desired in the particular treatment of a patient, it is now also possible with the method according to the invention to load a first peptide during cultivation of first batch of immature dendritic cells in the presence of a compound capable of inducing maturation of said immature dendritic cells, and to load a second peptide during cultivation of a second batch of immature dendritic cells, and mix the thus obtained batches of mature dendritic cells, each loaded with a different type of antigen, in the appropriate ratio.

Although the skilled person may, based on the current disclosure easily determine for each peptide the optimal amount per milliliter of medium that is present during the cultivating of the immature dendritic cells, in a preferred embodiment of the method between 0,1-80 microgram, 1-80 microgram, preferably 5-50 microgram, more preferably 10-40 microgram, of the peptide, e.g. antigen, is provided per ml culture medium. In case during cultivating the medium is replaced, or medium is added, it is preferred to, at the same time also re-add the peptide in order to provide for the above-mentioned amounts of peptide per milliliter of medium used.

Alternatively, between 0,1-80 microgram, 1-80 microgram, preferably 5-50 microgram, more preferably 10-40 microgram, of the peptide, e.g. antigen, is provided per 0.2-1*10̂6 (i.e. 200,000-1,000,000 cells) of immature dendritic cells that are provided at the start of the cultivating in the presence of a compound capable of inducing maturation of immature dendritic cells. In other words, when the peptide is added to the immature dendritic cells after some time during cultivating under conditions that induce the maturation of said immature dendritic cells, it is preferred to add about 1-80 microgram, preferably 5-50 microgram, more preferably 10-40 microgram, of the peptide, e.g. antigen, per 0.2-0.4*106 (i.e. 200,000-400,000 cells) of immature dendritic cells that are provided at the start of the said cultivating.

In other embodiment there is provided for a method according to the invention wherein the immature dendritic cells of step a) are obtained by cultivating CD34+ cells in the presence of at least one compound, preferably at least two, three or more compounds, capable of inducing differentiation of said CD34+ cells into immature dendritic cells, and preferably wherein during at least part of the period of said cultivating an anthracycline and/or an anthracenedione is provided to the culture medium.

European Patent application EP2281030 discloses that growing time of CD34 positive cells in the presence of a compound capable of inducing differentiation of said CD34 positive cells into immature dendritic cells, can be shortened (time until immature dendritic cells are obtained) by providing for at least part of the period of cultivating, an anthracycline and/or an anthracenedione to the culture medium.

For example, the anthracycline and/or an anthracenedione is selected from the group consisting of daunorubicin, doxorubicin, pirarubicin, aclarubicin, epirubicin, oxaunomycin, andidarubicin and mitoxantrone.

The compound which is capable of inducing differentiation of the cells is preferably selected from the group consisting of GM-CSF, TNF-alpha, IL-4 and TGF-beta 1 as documented herein. In such method according to the invention, the at least one compound capable of inducing maturation of the dendritic cells, is preferably selected from the group consisting of TNF-alpha, IL-6, PGE2 or IL-1 Beta, and as documented herein. However, the current invention is not limited to any particular (combination of) compounds. Other suitable combinations of compounds that are able to induce differentiation are well-known to the skilled person.

For example, in case of CD34 positive cells like DCOne cells, MUTZ3 cells, human cells, or human tumor cells the cells are, in one embodiment, contacted with from 0.05 nM to 20 nM mitoxantrone and/or from 10 to 120 nM doxorubicin, in the presence of from 50 to 150 ng/ml GM-CSF, from 5 to 20 ng/ml IL-4 and from 0,5 to 4 ng/ml TNF-alpha or wherein the cells are contacted with from 0.05 nM to 20 nM mitoxantrone and/or from 10 to 120 nM doxorubicin, in the presence of from 5 to 20 ng/ml TGF-beta 1, from 50 to 150 ng/ml GM-CSF, and from 0.5 to 4 ng/ml TNF-alpha.

Thus by combining both cultivating of the progenitors cells in the presence of differentiation medium to which such anthracycline and/or an anthracenedione is added, followed by cultivating the immature dendritic cells in the presence of a maturation medium to which a peptide to be loaded by the dendritic cells is added, allows for an improved and efficient method for providing mature dendritic cells loaded with an exogenously added peptide, in a shorter time period and with less change on cultivating errors and/or problems like infection or cell death. This is in particular preferred when using cells of a cell line, in particular for DCOne cells.

In a preferred embodiment the provided immature dendritic cells are cultivated in a maturation medium (i.e. a medium capable of inducing maturation of the immature dendritic cells e.g. by comprising compounds capable of inducing such maturation) comprising a combination of one or more compounds capable of inducing maturation, alone, or in combination, preferably selected from the group consisting of those documented herein, in particular TNF-alpha, PGE-2, IL-6 and IL-1beta.

Other important maturation inducing compounds, in addition to those already documented herein, are TLR ligands (Toll-like receptor ligands), which are wide available from various suppliers (e.g. from Imgenex www.imgenex.com) and CD40 ligand.

As already documented above, preferred compounds capable of inducing differentiation of progenitor cells, in particular CD34 positive cells, e.g CD34 positive cells of a cell line, towards immature dendritic cells include those documented herein, in particular, those selected from the group consisting of GM-CSF, TNF-alpha, IL-4 or TGF-beta1, or combinations thereof.

It was found that the period of peptide loading during the maturation of the immature dendritic cells towards mature dendritic cells may be any suitable time. In particular it is preferred that the peptide is provided to the maturation medium (i.e. the medium comprising compounds capable of inducing maturation of the immature dendritic cells) for a period of between 1-48, preferably for a period of between at least 2-30 hours, even more preferably for a period of between 3-20 hours, even more preferably for a period of between 3-10 hours.

In a preferred embodiment, the peptide is provided at least 24, 19, 10, 9, 8, 7, 6, 5, 4, 3 hours before the end of the cultivation in the presence of maturation medium, i.e. in the presence of compounds that are capable of inducing maturation of the immature dendritic cells, is ended.

In another embodiment, the peptide is provided to the cells at least for 10%, more preferably at least 50%, even more preferably at least 75%, most preferably at least 90% of the total time of maturation of the cells in the maturation medium, i.e. in the presence of compounds that are capable of inducing maturation of the immature dendritic cells.

In a preferred embodiment, the peptide is added to the maturation medium at a moment at least 5%, at least 10%, at least 25%, or at least 50% before the end of the cultivation in the maturation medium. For example in case the maturation in total is 10 hours, adding the peptide(s) one hour before the end of the maturation (i.e. after 9 hours of cultivation) is the moment 10% before the end of the cultivation in the maturation medium.

Preferably, the loading of the peptide is started at the same time as the maturation of the cells, and for the whole period of maturation.

In a further embodiment the mature dendritic cells loaded with an exogenously added peptide are irradiated.

Irradiation can for example be achieved by gamma irradiation at 30-150, e.g. 100 Gy for a period of 1 to 3 hours, using a standard irradiation device (Gammacell or equivalent).

It was found that with the method according to the invention, the mature dendritic cells loaded with the exogenously added peptide are not negatively influenced by said irradiation treatment.

Irradiation, in particular of the mature dendritic cells obtained from a cell line, ensures that any remaining progenitor cell, in particular CD34 positive cell, present, for example, in the initially provided immature dendritic cells cannot continue dividing. The cells may, for example, be irradiated prior to injection into patients, when used as a vaccine, or immediately after cultivating is stopped.

According to a last embodiment, there is provided for a method according to the invention the mature dendritic cells loaded with a exogenously added peptide are stored at a temperature below 0° C., preferably below −150° C., preferably in a medium that is suitable for direct injection into a human subject, preferably a freezing medium comprising no more than 15%, preferably no more than 10%, 5% or 2% DMSO, for example such as provided by BiolifeSolution under the trade name Cryostor (http://biolifesolutions.com/), or any other suitable freezing medium.

A skilled person will understand the term freezing medium as a medium suitable for freezing cells while mainly preserving the structural integrity of the cells, allowing for post-thaw viability, recovery, and/or functioning of the mature dendritic cells that were stored in such medium.

It was surprisingly found that with the method according to the current invention, mature dendritic cells loaded with exogenously added peptide may be obtained that may be stored at temperatures below 0° C., preferably below −150° C., for extended periods (e.g. up to a year of even more), without substantial loss of functionality or substantial loss of peptides loaded by the method according to the invention.

According to another aspect of the current invention there is provided for a a pharmaceutical composition comprising mature dendritic cells loaded with an exogenously added peptide and stored in a medium comprising no more than 15%, preferably no more than 10%, 5% or 2% DMSO, for example Cryostor, preferably wherein the pharmaceutical composition is stored at a temperature below 0° C., preferably wherein the exogenously added peptide is a tumor antigen, even more preferably wherein the mature dendritic cells are obtained from a cell line.

Also provided is for the use of the pharmaceutical composition obtainable by the method according to the invention, and in particular as document above, in the treatment of a various diseases such as cancers.

### EXAMPLES

The examples have been performed with both primary cells and with cell lines. Cell lines are preferred.

### Example 1

### Differentiation of CD34 Positive Progenitor Cells

CD34 positive progenitors cells, for example MUTZ3 or DCOne cells, were differentiated into immature DC, e.g. immature MUTZ3 or DCOne derived immature DC, by culturing the cells for 3 days at a concentration of 0.3*10E6 cells/ml in culture medium (MEM-α with 10% FCS and penicillin/streptomycin) supplemented with 500 IU/ml GM-CSF, 10 ng/ml IL-4, 240 IU/ml TNF-α and 2 nM mitoxantrone. Alternatively, cells can be differentiated into immature DC in 6 days in the same culture medium without mitoxantrone. After differentiation, cells are harvested and the DC phenotype is determined with FACS analysis. For example, immature MUTZ3 DC will express CD1a, which is absent on the progenitors. Also other DC markers are expressed like CD40, CD80 and CD86, whereas the maturation marker CD83 is absent. Immature DCOne derived dendritic cells express CD1a, CD40, CD80 and CD86, whereas the maturation marker CD83 was absent.

Alternatively, CD14+ cells were isolated from patients PBMC by immunomagnetic separation. Subsequently cells were cultured for 5-7 days in medium (CellGro/RPMI/X-VIVO 15 etc with FCS/HPS) medium supplemented with 10-100 ng/ml GM-CSF and 10-50 ng/ml IL-4. After differentiation cells were matured by the addition (or replacement of the medium) of a maturation cocktail consisting of different maturation cytokines (e.g. 20 ng/ml TNF-α, 10 ng/ml IL-1β, 10 μg/ml PGE-2) or TLR ligands. (Raïch-Regué et al, Vaccine 2012; Chiang et al., JTM 2011; Sadallah et al. JI 2011)

### Example 2

### Maturation of Immature Dendritic Cells

After differentiation, immature DC, for example immature MUTZ3 or DCOne derived immature DCs, were matured into mature DC by culturing the cells for 24-48 hours in MEM-alpha medium containing 10% FCS, 50 ng/ml TNF-alpha, 25 ng/ml IL-1β, 100 ng/ml IL-6 and 1 μg/ml PGE2. After maturation, DC were harvested and the phenotype was determined by FACS analysis. Mature DC can be discriminated from immature DC by expression of the maturation marker CD83 and increased expression of the co-stimulatory molecule CD80.

### Example 3

### Peptide Loading of Immature Dendritic Cells

MUTZ3 derived immature dendritic cells or DCOne immature dendritic cells (1×10E6 cells) were loaded with peptides during maturation. For this, peptides were added at a final concentration between 1 and 30 μg/ml of peptide between 19 and 4 hours before the end of the maturation in the presence or absence of 3 μg/ml β2-microglobulin. After loading, cells were harvested and the phenotype was determined by FACS analysis.

As a control, mature DC were loaded after maturation. Cells were harvested and after washing, the cells were resuspended at a concentration of 1*10E6 cells/ml. A final concentration of 10 μg/ml peptide and 3 μg/ml β2-microglobulin was added to the cells. Hereafter, cells were incubated for 2 hours at 37° C. After loading, cells were harvested and the phenotype was determined by Flow cytometry (FACS) analysis.

Many different protocols for loading autologous DC (e.g. obtained from peripheral blood monocytes) with peptides have been described. As a second control, cells were harvested after maturation and mature DC were pulsed for 1-4 h at 37° C. in a humidified atmosphere of 5% CO2 with different concentrations. Afterwards cells were harvested, washed once with medium, and then immediately used (2 references: Knippertz et al., Int. J. Hyperthermia, 2011; Hangalapura et al., JIT 2010).

### Example 4

### Irradiation of Mature DCs

After maturation, mature DC, e.g. mature MUTZ3 DC or DCOne derived mature DC, were harvested and irradiated with 50 Gy in a Gamma source. After irradiation the cells were washed and stored in freezing medium in liquid N2. Irradiation did not influence the stability of the peptide-MHC complex nor the loading efficiency, nor did it affect the capacity to activate T cells. Similar results were observed after a freeze/thaw cycle.

### Example 5

### Prior Art Peptide Loading of Mature Dendritic Cells (After Maturation)

In the control experiments, mature DCs were loaded with peptides according to prior art methods. In order to load mature dendritic cells with MART-1 peptide, the CD34 positive cells were differentiated and matured into functional DC as described above, and subsequently loaded for 2 hours at 37° C. with the MART-1 peptide.

The efficiency of peptide loading was assessed by the ability of peptide loaded DCs to activate MART-1 specific T-cells. Therefore, MART-1 loaded DCs were co-cultured for 4 hours with T-cells from a MART-1 specific CTL clone (Bontkes (2005) Hum Immunol. 66(11):1137-45), whereafter the percentage of IFN producing cells (marker for T-cell activation) was determined. To determine the priming capacity of MART-1 peptide loaded DCs cells, irradiated MART-1 peptide loaded DC cells were co-cultured with CD8+ T-cell from a healthy donor for 1 week. Each week, the percentage of MART-1 tetramer-positive CD8+ cells was determined where after the T-cells were re-stimulated with MART-1 peptide loaded DCs. As a control unloaded DCs were taken along, incapable of inducing the proliferation of MART-1 positive T-cells.

DCs presenting the MART-1 peptide were found capable of inducing the production of IFN in 50% of the MART-1 specific CTL. In addition, DCs loaded with MART-1 peptide were capable to prime MART-1 specific T-cells in a healthy donor and induce proliferation of these MART-1 specific T-cells, as determined by the percentage of MART-1-tetramer (Tm)-positive cells among CD8 positive T cell bulk cultures.

These data demonstrate that the peptide loading approach as described in the literature is sufficient to load the mature DCs and results in peptide loaded DCs that are fully functional with respect to the capacity to prime and activate tumour antigen-specific CD8+ T cells.

### Example 6

### Loading of DCs with Peptide during Maturation of the DCs

Although the above described loading procedure is suitable for the loading of peptides on mature DCs, this approach is not preferred for large scale production of peptide loaded DCs. This procedure requires loading of the peptide in a small volume, which is difficult for large scale production as, for large scale production, peptide loading in a small volume will be time-consuming. Also, since mature dendritic cells are not able to proliferate, their life span after maturation is limited. Therefore, it is preferred to keep the time between maturation and final formulation (irradiation, fill & finish) of a dendritic cell vaccine product as short as possible. This requirement is addressed by the present invention.

According to an embodiment of the present invention, MART-1 peptides were added during the maturation of DCs instead of loading the cells with peptides after maturation. This approach is easily applicable in GMP based culture processes used for production of the clinical batches of a dendritic cell based vaccine.

Different concentrations of MART-1 peptide (0, 10, 20 and 30 μg/ml) were added at the start of the maturation of the immature DCs. The cells were allowed to mature for 24 hours and cells were analyzed for the amount of presented peptide, the viability and the phenotype. The amount of MHC-MART-1 peptide complex present on DCs was determined using a MART-1 specific soluble PE-labeled TCR antibody, that is commercially available (TCR MART-1 PE: PE-labeled MART-1:26-35(27L) STAR™ Multimer alter Bioscience). This antibody recognizes the MART-1 peptide bound to HLA-A2 and using this TCR one can specifically determine the percentage of cells presenting the MART-1 peptide and allows a more exact quantification of peptide-MHC complexes as compared to the T-cells clones.

It was found that dendritic cells may be efficiently loaded during maturation and the peptide loading was dose-dependent. Using a peptide concentration of 20 μg/ml, it was found that more than 60% of the cells presented the MART-1-peptide, whereas 80% of cells were positive when cells were loaded with 30 μg/ml of peptide. Loading of peptide on immature DCs during their maturation did not affect the viability of the DCs cells. For comparison, mature dendritic cells were loaded for 2 hours with 10 μg/ml MART-1 peptide and this resulted in 60% of peptide-presenting mature DCs

As it might be possible that peptide loading during maturation would affect the ability of DCs to mature, the phenotype of the DCs after loading with MART-1 peptide was determined. Peptide loading during maturation did not influence the ability of immature DCs to mature as the phenotype of the mature DCs was similar in all conditions tested.

Based on these results it was concluded that peptide concentrations of 20 and 30 μg/ml are examples of suitable concentrations that result in efficient loading of mature DCs without affecting the viability and phenotype of cells and with acceptable recovery rates.

A major aspect in the development of peptide loaded DC vaccines is the stability of the MHC-peptide complex as the complex may dissociate depending on the binding affinity of the epitope. Furthermore, for production or industrial manufacturing purposes it is essential to establish the time window during which peptides can be added during the maturation without affecting the loading efficiency. Therefore experiments were performed in which MART-1 peptide (20 μg/ml) peptide was added at different time points during maturation, ranging from the start of the maturation (t=25) to 2 hours before the end of the maturation (t=2). The results demonstrate that the highest percentage of peptide-presenting DCs may be achieved by adding the peptide between 2 and 19 hours before the end of the maturation. Adding the peptide at different time points during maturation did not result in an effect on the viability or recovery of the DCs that was respectively above 80% and 60% in all cases.

### Example 7

### Effect of Freeze/Thawing on the Stability of the MHC-Peptide Complex

As a vaccine comprising mature dendritic cells would preferably be developed as an off-the shelf vaccine, it is essential that peptides remain bound after freeze/thawing. Mature dendritic cells loaded with exogenously added peptide obtained in a method according to the invention were frozen in Cryostor preservation media, having DMSO content as disclosed herein in general for freezing media, and subsequently thawed. The results showed that a freeze/thaw cycle does not affect the amount of peptide bound, indicating that the peptide loaded DC obtained by the method according to the invention is suitable for off-the shelf applications. This was particularly evident when cells from the DCOne cell line were used in a method according to the invention.

1. A method for obtaining mature dendritic cells loaded with an HLA Class I antigenic peptide, the method comprising:
cultivating immature dendritic cells in a culture medium in the presence of at least one compound that is capable of inducing maturation of the immature dendritic cells, and wherein during at least part of the cultivating at least one exogenously added HLA Class I antigenic peptide is present in the culture medium, wherein the peptide consists of 8, 9, 10 or 11 amino acids, and
collecting the mature dendritic cells loaded with an HLA Class I antigenic peptide.

2. The method according to claim 1, wherein the immature dendritic cells are obtained from progenitors cells of a CD34+ cell line.

3. The method according to claim 2, wherein the cell line is selected from the group consisting of KG1, THP-1, HL-60, K562, U-937, MUTZ3 or a DCOne cell line, deposited at the DSMZ under accession number DSM ACC3189 or cell lines derived thereof.

4. The method according to claim 1, wherein the peptide is an antigenic peptide or a tumor antigen

5. The method according to claim 4 wherein the antigenic peptide is selected from the group consisting of WT-1, NY-ESO, MAGE-A3, Mart-1, NY-ESO, PRAME, RHAMM, PSA, PSMA, Her2Neu and MUC-1.

6. The method according to claim 1, wherein the peptide comprises an amino acid sequence selected from the group consisting of SEQ ID NOs: 1-7.

7. The method according to claim 1, wherein the immature dendritic cells are obtained by cultivating CD34+ cells in the presence of at least one compound capable of inducing differentiation of the CD34+ cells into immature dendritic cells and wherein during at least part of the cultivating an anthracycline and/or an anthracenedione is provided to the culture medium.

8. The method according to claim 1, wherein the compound capable of inducing maturation of immature dendritic cells is selected from the group consisting of TNF-alpha, IL-6, PGE2, and IL-1beta.

9. The method according to claim 7, wherein the compound capable of inducing differentiation of the CD34+ cells is selected from the group consisting of GM-CSF, TNF-alpha, IL-4 and, TGF-beta1.

10. The method according to claim 1, wherein the peptide is provided to the medium during maturation of the immature dendritic cells for a period of between 1 and 48 hours, between 2 and 30 hours, between 3 and 20 hours, or between 3 and 10 hours.

11. The method according to claim 1, further comprising irradiating the mature dendritic cells loaded with an exogenously added peptide.

12. The method according to claim 1, further comprising storing the mature dendritic cells loaded with an exogenously added peptide in a medium at a temperature below 0° C.

13. A pharmaceutical composition comprising mature dendritic cells obtained by the method according to claim 1.

14. A method for the treatment of cancer, inflammatory and infectious diseases and autoimmune diseases in a subject, the method comprising:
administering to a subject identified as suffering from cancer, an inflammatory disease, an infectious disease, or an autoimmune diseases the pharmaceutical composition of claim 13.

15. A method for activating cytotoxic T-cells specific for a tumor antigen in vitro, the method comprising:
contacting a mature DC obtained by the method according to claim 1 with a population of T-cells.

16. The method according claim 8, wherein the compound capable of inducing differentiation of the CD34+ cells is selected from the group consisting of GM-CSF, TNF-alpha, IL-4 or TGF-beta1.

17. The method according to claim 15, wherein the T-cell are CD8+ T-cells.

18. The method according to claim 12, wherein the medium is suitable for direct injection into a human subject

19. The method according to claim 18, wherein the medium is Cryostor.

20. The method according to claim 1, further comprising administering to a subject identified as suffering from cancer, an inflammatory disease, an infectious disease, or an autoimmune diseases the mature dendritic cells loaded with an HLA Class I antigenic peptide.
